SEQUENCE LISTING (46 sequences)

SEQ ID NO: 1 - protein, 10 aa, Artificial Sequence
  TAU PEPTIDE
TPSLPTPPTR

SEQ ID NO: 2 - protein, 12 aa, Artificial Sequence
  TAU PEPTIDE
LQTAPVPMPDLK

SEQ ID NO: 3 - protein, 16 aa, Artificial Sequence
  TAU PEPTIDE
IGSLDNITHVPGGGNK

SEQ ID NO: 4 - protein, 17 aa, Artificial Sequence
  TAU PEPTIDE
IGSLDNITHVPGGGNKK

SEQ ID NO: 5 - protein, 5 aa, Mouse
TREPK

SEQ ID NO: 6 - protein, 8 aa, Artificial Sequence
  TAU PEPTIDE
YVSSVTPR

SEQ ID NO: 7 - protein, 12 aa, Artificial Sequence
  TAU PEPTIDE
VQIVYKPVDLSK

SEQ ID NO: 8 - protein, 9 aa, Artificial Sequence
  TAU PEPTIDE
VAVVRTPPK

SEQ ID NO: 9 - protein, 10 aa, Artificial Sequence
  TAU PEPTIDE
TPSLPTPPTR

SEQ ID NO: 10 - protein, 10 aa, Artificial Sequence
  TAU PEPTIDE
TPPSSGEPPK

SEQ ID NO: 11 - protein, 16 aa, Artificial Sequence
  TAU PEPTIDE
TPPAPKTPPSSGEPPK

SEQ ID NO: 12 - protein, 10 aa, Artificial Sequence
  TAU PEPTIDE
TDHGAEIVYK

SEQ ID NO: 13 - protein, 20 aa, Artificial Sequence
  TAU PEPTIDE
STPTAEDVTAPLVDEGAPGK

SEQ ID NO: 14 - protein, 20 aa, Artificial Sequence
  TAU PEPTIDE
STPTAEDVTAPLVDEGAPGK

SEQ ID NO: 15 - protein, 30 aa, Artificial Sequence
  TAU PEPTIDE
STPTAEAEEAGIGDTPSLEDEAAGHVTQAR

SEQ ID NO: 16 - protein, 30 aa, Artificial Sequence; identical to SEQ ID NO: 15
  TAU PEPTIDE

SEQ ID NO: 17 - protein, 11 aa, Artificial Sequence
  TAU PEPTIDE
SPVVSGDTSPR

SEQ ID NO: 18 - protein, 11 aa, Artificial Sequence
  TAU PEPTIDE
SPVVSGDTSPR

SEQ ID NO: 19 - protein, 15 aa, Artificial Sequence
  TAU PEPTIDE
SGYSSPGSPGTPGSR

SEQ ID NO: 20 - protein, 15 aa, Artificial Sequence
  TAU PEPTIDE
SGYSSPGSPGTPGSR

SEQ ID NO: 21 - protein, 18 aa, Artificial Sequence
  TAU PEPTIDE
QEFEVMEDHAGTYGLGDR

SEQ ID NO: 22 - protein, 39 aa, Artificial Sequence
  TAU PEPTIDE
QAAAQPHTEIPEGTTAEEAGIGDTPSLEDEAAGHVTQAR

SEQ ID NO: 23 - protein, 12 aa, Artificial Sequence
  TAU PEPTIDE
LQTAPVPMPDLK

SEQ ID NO: 24 - protein, 9 aa, Artificial Sequence
  TAU PEPTIDE
LDLSNVQSK

SEQ ID NO: 25 - protein, 10 aa, Artificial Sequence
  TAU PEPTIDE
KLDLSNVQSK

SEQ ID NO: 26 - protein, 21 aa, Artificial Sequence
  TAU PEPTIDE
KDQGGYTMHQDQEGDTDAGLK

SEQ ID NO: 27 - protein, 21 aa, Artificial Sequence
  TAU PEPTIDE
KDQGGYTMHQDQEGDTDAGLK

SEQ ID NO: 28 - protein, 8 aa, Artificial Sequence
  TAU PEPTIDE
IGSTENLK

SEQ ID NO: 29 - protein, 23 aa, Artificial Sequence
  TAU PEPTIDE
ESPLQTPTEDGSEEPGSETSDAK

SEQ ID NO: 30 - protein, 23 aa, Artificial Sequence
  TAU PEPTIDE
ESPLQTPTEDGSEEPGSETSDAK

SEQ ID NO: 31 - protein, 43 aa, Artificial Sequence
  TAU PEPTIDE
DQGGYTMHQDQEGDTDAGLKESPLQTPTEDGSEEPGSETSDAK

SEQ ID NO: 32 - protein, 20 aa, Artificial Sequence
  TAU PEPTIDE
DQGGYTMHQDQEGDTDAGLK

SEQ ID NO: 33 - protein, 20 aa, Artificial Sequence
  TAU PEPTIDE
DQGGYTMHQDQEGDTDAGLK

SEQ ID NO: 34 - protein, 24 aa, Artificial Sequence
  TAU PEPTIDE
AEEAGIGDTPSLEDEAAGHVTQAR

SEQ ID NO: 35 - protein, 14 aa, Artificial Sequence
  TAU PEPTIDE
AAFPGAPGEGPEAR

SEQ ID NO: 36 - DNA, 402 bp, MOUSE
atgaaatgcagctgggtcatcttcttcctgatggcagtggttataggggtcaattcagaggttcagctgcagcagtctggggctgagcttgtgaggccgggggcctcagtcaagttgccctgcacagcttctggctttaacattaaagacgactatatgaactgggtgatgcagaggcctgaacggggcctggagtggattggatggattgatcctgagaatggtgatactgcatatgcctcgaagttccagggaaaggccactatgactgcagacacatcctccaacacagcctacctgcagctcagcagcctgacatctgaggacactgccgtctattactgtacttcaggtggtcgtttcgactactggggccaaggcacctctctcacagtctcctca

SEQ ID NO: 37 - protein, 134 aa, MOUSE
MKCSWVIFFLMAVVIGVNSEVQLQQSGAELVRPGASVKLPCTASGFNIKDDYMNWVMQRPERGLEWIGWIDPENGDTAYASKFQGKATMTADTSSNTAYLQLSSLTSEDTAVYYCTSGGRFDYWGQGTSLTVSS

SEQ ID NO: 38 - DNA, 393 bp, MOUSE
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaagcacctatttagaatggtacctgcagaaaccaggccagtctccaaagctcctgatctacaaagtttccaaccgcttttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaa

SEQ ID NO: 39 - protein, 131 aa, MOUSE
MKLPVRLLVLMFWIPASSSDVLMTQTPLSLPVSLGDQASISCRSSQSIVHSNGSTYLEWYLQKPGQSPKLLIYKVSNRFSGVPDRFSGSGSGTDFTLKINRVEAEDLGVYYCFQGSHVPWTFGGGTKLEIK

SEQ ID NO: 40 - protein, 10 aa, MOUSE
GFNIKDDYMN

SEQ ID NO: 41 - protein, 17 aa, MOUSE
WIDPENGDTAYASKFQG

SEQ ID NO: 42 - protein, 6 aa, MOUSE
GGRFDY

SEQ ID NO: 43 - protein, 16 aa, MOUSE
RSSQSIVHSNGSTYLE

SEQ ID NO: 44 - protein, 7 aa, MOUSE
KVSNRFS

SEQ ID NO: 45 - protein, 9 aa, MOUSE
FQGSHVPWT

SEQ ID NO: 46 - protein, 7 aa, MOUSE
PPTREPK

(19) United States
(12) Patent Application Publication
(10) Pub. No.: US 2015/0253341 A1
(43) Pub. Date: Sep. 10, 2015

(54) QUANTIFICATION OF TAU IN BIOLOGICAL SAMPLES BY IMMUNOAFFINITY ENRICHMENT AND MASS SPECTROMETRY

(71) Applicant: Merck Sharp & Dohme Corp., Rahway, NJ (US)

(72) Inventors: Thomas McAvoy, Millington, NJ (US); Michael E. Lassman, Scotch Plains, NJ (US); Omar F. Laterza, New York, NY (US)

(73) Assignee: MERCK SHARP & DOHME CORP., Rahway, NJ (US)

(21) Appl. No.: 14/615,480

(22) Filed: Feb. 6, 2015

Related U.S. Application Data
(60) Provisional application No. 61/937,954, filed on Feb. 10, 2014.

(51) Int. Cl. G01N 33/68; C07K 14/47

(52) U.S. Cl. CPC G01N 33/6896; C07K 14/4711; G01N 2333/4709; G01N 2458/15; G01N 2800/2821; G01N 2333/976

(57) ABSTRACT

The present invention provides a quantitative immunoaffinity LC-MS/MS assay for detection and quantification of Tau protein in a biological sample.

### FIELD OF THE INVENTION

The present invention relates to the field of clinical biochemistry and provides a bioanalytical immunoaffinity-based mass spectrometry (IA-MS) assay for the quantification of Tau in a biological sample.

### BACKGROUND OF THE INVENTION

Substantial efforts are currently ongoing to develop and characterize biomarkers that have the potential to aid in clinical diagnosis of tauopathies, including AD, and the development of new therapeutics. van Gool & Hendrickson (2012) Expert Rev. Proteomics 9:165-79. For example, published AD research has focused on understanding the molecular changes that occur with disease in the plaque-forming amyloid beta (Aβ) peptide (Mattsson et al. (2012) Biomark. Med. 6:409-17) as well as Tau (Andreasson et al. (2012) Biomark Med 6:377-89).

Traditionally, levels of Aβ peptides and Tau proteins have been measured in CSF, which is in direct contact with the brain and as a consequence may best reflect biochemical processes in this organ. Tau protein present in the cerebrospinal fluid (CSF) represents a predominantly intracellular protein that is also found in the extracellular space of the central nervous system. Tau is preferentially expressed in neurons throughout the nervous system and plays an important role in the regulation of microtubule stability and axon structure. Tau aggregation is a hallmark of a series of neurodegenerative diseases, including Alzheimer's disease (AD), known as tauopathies. It is thought that, when Tau is hyperphosphorylated primarily by the action of serine/threonine kinases, it dissociates from microtubules, leading to the formation of paired helical filaments (PHFs) which, in turn, aggregate into neurofibrilary tangles (NFTs) and neuropil threads. Hanger et al. (2009) Trends Mol. Med. 15:112-9. It is hypothesized that the formation of NFTs leads to neuron and synapse loss, ultimately contributing to the development of dementia. Hanger et al. (2009) Trends Mol. Med. 15:112-9.

In addition, Tau protein released into the CSF is related to the extent of NFT pathology (Rosenmann (2012) J. Mol. Neurosci. 47:1-14, Tapiola et al. (2009) Arch. Neurol. 66:382; Buerger et al. (2006) Brain 129 (Pt 11):3035) and brain tissue loss (Whitwell et al. (2008) Neurology 71:743) and as a consequence extracellular Tau has been measured in human clinical samples for more than a decade. There are six isoforms of Tau of varying length (352, 381, 383, 410, 412, 441AA), many post-translational modifications, and an unknown number of potential degradation products circulating in CSF. It has been predicted that Tau modifications (including, but not limited to, phosphorylation, ubiquination, oxidation, truncation, prolyl isomerization, glycation, glycosylation, nitrosylation and others) may be related to the disease progression. Cohen et al. (2011) Nat. Commun. 2:252. Similarly, changes in isoform distribution (alternative splicing) of Tau protein in the CSF may also signify changes in the pathological processes associated with neurodegeneration. The existence of multiple isoforms and numerous degradation products makes Tau a challenging protein to quantitate accurately.

There are multiple immunoassays for the quantification of Tau in CSF. Kang et al. (2013) Clin. Chem. 59:903-16. Though sensitive, immunoassays have inherent limitations in the number of sub-species of a give protein that can be detected with a single assay. In addition, given the molecular diversity of Tau in CSF including truncations and post-translational modifications, the true selectivity of Tau immunoassays is unknown. There remains an unmet need for a sensitive and highly specific assay for the detection, molecular characterization and quantification of Tau in biological samples.

### SUMMARY OF THE INVENTION

The present invention provides a sensitive and highly selective immunoaffinity LC-MS/MS (IA-MS) assay for Tau. As shown herein, the disclosed assay provides a clinically viable method to quantitate Tau by mass spectrometry and provides a value bioanalytical tool for elucidating the role of CSF Tau in the progression of tauopathies, including but not limited to Alzheimer's disease.

The bioanalytical mass spectrometric assay disclosed herein employs an immunoaffinity enrichment step using an anti-Tau monoclonal antibody to capture the Tau polypeptides present in the biological sample prior to trypsin digestion. The anti-Tau monoclonal antibody binds to a region shared among all Tau isoforms (TREPK) (SEQ ID NO: 5) that is directly adjacent to, and partially overlapping with, the surrogate peptide analyte (TPSLPTPPTR) (SEQ ID NO:1). For detection, the LC-MS/MS Trizaic nanoTile system was found to be sufficiently robust for clinical analysis, with fast cycle times, reproducible peptide retention times and no need for user intervention during the course of hundreds of sample analyses.

Both ligand binding assays and mass spectrometry (MS) are routinely used in clinical analysis. The Tau quantification method disclosed herein combines the selectivity of immunoaffinity (IA) extraction with the specificity of MS detection (IA-MS). The precision and accuracy of the assay disclosed herein exploits the orthogonal resolving power of chromatographic separation followed by MS/MS detection thereby facilitating the detection of any isoform, or modified form of Tau. As shown herein immunoaffinity enrichment followed by LC-MS/MS analysis provides sufficient sensitivity to enable mass spectrometry-based detection and quantification of specific tryptic Tau peptides present in biological samples, including human CSF clinical samples. The lower limit of quantitation of the assay is 10 pg/mL or 0.25 pM.

As shown herein, the low CSF concentrations of Tau in healthy and many diseased subjects requires the use of advanced techniques in sample preparation and analysis, such as the use of immunoaffinity enrichment for concentration of Tau protein, as well as the use of robust UPLC microflow LC-MS/MS to enable measurements of low abundance peptides. The mass spectrometry based method disclosed herein provides an orthogonal technique that can confirm the selectivity of more commonly used immunochemistry-based assays, as well as a powerful platform that can be applied to measure specific modifications of Tau.

The disclosed mass-spectrometry-linked immunological method disclosed herein employs an anti-human Tau specific monoclonal antibody (mAb) to selectively enrich Tau from routinely available volumes of CSF (up to 1 mL) followed by tryptic digestion to produce proteotypic peptide fragments that can be quantified by LC-MS/MS. The disclosed method provides an assay that can be used to measure total-Tau, and other post-translationally modified forms of Tau in biological samples. The IA-MS assay of this invention provides an important tool for evaluation of the efficacy of therapeutic drug candidates, and for clinical use as in vitro diagnostic focused on both quantitative changes and modifications of Tau proteins in patient samples.

In one embodiment the invention provides a method for measuring Tau protein in a biological sample comprising the steps of: providing a CSF sample; adding an isotope labeled internal standard Tau protein to the CSF sample to produce a spiked CSF sample; contacting the spiked CSF sample with an anti-Tau antibody that is covalently conjugated to solid phase particles; maintaining the sample under conditions suitable to allow the anti-Tau antibody to bind to Tau protein present in the sample; washing the particles; eluting the Tau protein from the particles; contacting the recovered protein with trypsin under conditions suitable to digest the protein into an analyte sample comprising a composition of Tau peptides; and performing tandem mass spectrometry to detect and measure the amount of surrogate Tau peptide and isotope labeled Tau peptide present in the analyte sample. The use of microflow-chromatography, combined with a microfluidic device, provides enhanced sensitivity with the robustness required for clinical assays. Zhou et al. (2013) Rapid Commun. Mass Spectrom. 27:1294-302.

In one embodiment the isotope labeled internal standard used in the assay is a heavy-isotope labeled recombinant human Tau protein, and the anti-Tau antibody is a monoclonal antibody specific for intact human Tau protein. Suitable isotope labels include, but are not limited to ²H, ¹³C, and ¹⁵N. These labels can be incorporated into an amino acid found within the Tau peptide analyte(s). The labeled amino acid is commonly lysine and arginine, but could be a different amino acid contained within the surrogate peptide sequence.

In practice, the method of the invention uses a surrogate Tau peptide to detect and measure the amount of Tau protein present in the biological sample. In various embodiments the surrogate Tau peptide is selected from the group of peptides defined by SEQ ID NOs: 1-4 and 6-35, e.g. SEQ ID NOs: 1-3, preferably SEQ ID NO: 1. A surrogate Tau peptide must be present in all Tau isoforms, and should be as free as possible from modifications, such as phosphorylation or oxidation, that would result in multiple masses of the peptide following digestion. It should produce a strong response signal on the mass spectrometer with sufficient sensitivity to quantitate endogenous levels from a biological sample.

In one embodiment the anti-Tau antibody used for the immunoaffinity enrichment of Tau is covalently linked to a solid phase. Suitable solid phases include paramagnetic particles, nonmagnetic particles, tube or well surfaces, and column substrate material.

In one embodiment, the anti-Tau antibody used for the immunoaffinity enrichment of Tau binds at an epitope that overlaps with the surrogate Tau peptide that is detected by mass spectrometry. In a particular embodiment the anti-Tau antibody comprises a variable heavy chain comprising the amino acid sequence set forth in SEQ ID NO.37 and a variable light chain comprising the amino acid sequence set forth in SEQ ID NO:39. In a particular embodiment the anti-Tau antibody binds to an epitope comprising or consisting essentially of the amino acid sequence set forth in SEQ ID NO:5 The method of the invention can be practiced using an anti-Tau antibody that is immobilized on a solid phase, for example, magnetic beads.

In an alternative embodiment the method of the invention can include an elution step is performed under acidic conditions and further wherein the method optionally comprises a solvent evaporation step.

A calibration curve is prepared by a serial dilution series of Tau standard from 50 pM to 0.39 pM. CSF samples are added to wells of a 96-well 2 ml Axygen plate and PBS is added if needed to bring the total volume up to 1 mL. QC samples at three different levels are also analyzed with each run. 50 μl 10% Tween-20 and 10 μl of 10 nM internal standard recombinant Tau are added to each well using a repeater pipet. For each well, 100 μl 13A6 antibody-coated beads are added using a 0.1 ml tip on an Eppendorf repeater pipet. The plate is then sealed and mixed at 1200 rpm at RT for 2H on a plate shaker. Using a magnetic capture device, beads are collected on the sides of each well for two minutes. Liquid is removed and beads are washed with 1 ml PBS. The plate is recapped with new mat and mixed at 1200 rpm for 30 sec to fully resuspend beads. The wash process is repeated two additional times with PBS for a total of three washes. Following aspiration of final wash, 100 μl 2% formic acid solution in water is added to each well and mixed at 1200 rpm 20 min at room temperature to elute proteins. Eluted proteins are transferred to a 2 ml deepwell plate and dried 2 h in speed vac at 45° C. 50 μl digestion solution (100 mM AMBIC containing 20 μg/mL trypsin) is added to each well of dried proteins and mixed at 37° C. overnight at 1200 rpm. The digested samples are moved to a 4° C. autosampler and analyzed by LC-MS/MS.

A Waters nanoAcuity system (Milford, Mass., USA) is used to load 8 μl of each digested sample onto an 85 μm Trizaic nanotile containing a trapping column at 4 μl/min for 5 min in 100% Buffer A (0.1% formic acid in water). Elution of bound proteins and chromatography is performed with a linear gradient from 5-50% Buffer B (0.1% formic acid in acetonitrile) over 4 min followed by a 1 min 50-90% gradient with a constant tile temperature of 65° C. The column wash is held at 90% Buffer B for 1.5 min then equilibrated at 10% Buffer B for 1.5 min.

Mass spectrometry is performed using a Waters Xevo-TQS mass spectrometer (Milford, Mass., USA). Instrument-specific parameters for collision energy are experimentally determined during infusion of synthetic peptides. A capillary voltage of 3.0 kV is used for all analyses. Resolution settings are optimized for signal/noise and are set as follows: LM 1 resolution=2.5, HM 1 resolution=15.1, Ion energy 1=0.2, LM 2 resolution=2.8, HM 2 resolution=15.5, Ion energy 2=0.8.

### BRIEF DESCRIPTION OF THE DRAWINGS

FIG. 1 provides a flow chart of IA-MS workflow. Heavy-isotope labeled Tau internal standard (“Tau IS”) is added to CSF samples containing Tau protein and captured using Tau-selective monoclonal antibodies conjugated to magnetic beads in the Immunoaffinity Enrichment step. Unbound sample components are washed away before bound components are released. Bound proteins are dried then enzymatically digested by trypsin into peptides in the Trypsin Digestion step. Unique peptides derived from Tau are then quantified in the sample by LC-MS/MS Analysis. The heavy-isotope labeled arginine residue is indicated by an asterisk (*) and bold face font in the sequence of the Tau IS.

FIG. 2 schematically compares the sequence of various Tau isoforms, and shows the locations of other sequence elements of interest. Regions of consensus between human Tau protein isoforms are aligned with regions of isoform-specific sequence shown with shaded boxes. The tryptic peptide used as the analyte in the assay of the present invention is shown in the black box. The sequence identified by epitope mapping as the binding site of mAb 13A6 is shown in the open box.

FIGS. 3A and 3B show results of analysis of CSF Tau in study samples. FIG. 3A represents a graphic distribution of total-Tau levels determined for control and AD groups as measured by the IA-MS assay. FIG. 3B provides a graphic representation of the correlation of CSF total-Tau values of the PrecisionMed sample set as measured by the MSD immunoassay and IA-MS assay, as described in greater detail at Example 6.

### DETAILED DESCRIPTION OF THE INVENTION

### Definitions

As used herein, the term “limit of detection” of “LoD” refers to the sensitivity of the assays at the lowest concentration that can be detected above a sample which is identical except for the absence of the Tau. The signal in the absence of Tau is defined as the “Background.” As used herein, the LoD for Tau was defined as >3 standard deviations above the mean of the background.

As used herein, the term “lower limit of quantification” or “LLOQ” refers to the sensitivity of the assay in combination with the coefficient of variability to indicate the lowest concentration that can be reliably and reproducibly differentiated from background. This limit typically defines the practical working range of the assay at the low end of sensitivity and is the concentration that delivers a coefficient of variability of <20% across ≧three measured values.

“Tau” and “Tau protein” refer, unless otherwise indicated, to human Tau protein as it exists in the human central nervous system in any of six isoforms of 352-441 amino acid residues. Goedert et al. (1989) Neuron 3:519-526. Table 1 provides the NCBI accession numbers for the six Tau isoforms, the sequences of which are hereby incorporated by reference.

TABLE 1
Human Tau Isoforms

| Isoform | Length | NCBI Accession |
|---|---|---|
| 4 | 352 | NP 058525.1 |
| 7 | 381 | NP 001190180.1 |
| 3 | 383 | NP 058518.1 |
| 8 | 410 | NP 001190181.1 |
| 5 | 412 | NP 001116539.1 |
| 2 | 441 | NP 005901.2 |

As used herein, the term “Tau protein” refers to any protein of the Tau protein family including, but not limited to, native Tau protein monomer of any one or all of the known isoforms, precursor Tau proteins, Tau peptides, Tau intermediates, metabolites, naturally occurring post-translationally modified Tau proteins, and Tau derivatives that can be antigenic, or antigenic fragments thereof. Fragments include less than the entire Tau protein provided that the fragment is antigenic and will cause antibodies or antibody binding fragments to react with the Tau fragment.

Tau is encoded by a single gene, MAPT, located on chromosome 17q21. MAPT is over 50 kb in size and comprises two haplotypes, H1 and H2, with multiple variants of each. Exons 9, 10, 11 are imperfect copies of an 18 amino acid sequence termed a “repeat,” and each exon encodes a microtubule binding motif. The “repeat” is separated by a 13 to 14 amino acid inter-repeat sequence. The various Tau isoforms are generated by alternative splicing (which is developmentally regulated) creating both high and low molecular weight isoforms. The six isoforms which occur in the central nervous system, are differentiated by the presence or absence of sequences encoded by MAPT exons 2, 3 and 10.

As used herein, the term “tauopathies” refers to age-related neurodegenerative diseases that are characterized by the presence of aggregates of abnormally phosphorylated Tau, intracellular neurofibrillary tangles and extensive neuronal loss in the brain. Tauopathies include, but are not necessarily limited to, Alzheimer's disease, sporadic corticobasal degeneration, progressive supranuclear palsy, Pick's disease, hereditary frontotemporal dementia, and parkinsonism linked to chromosome 17 (FTDP-17).

As used herein, the term “surrogate Tau peptide” refers to proteolytic peptides produced from the digestion of Tau that can be analyzed/quantified as representative of the intact protein.

As used herein, the terms “Tau internal standard” or “Tau IS” refer to purified recombinant Tau of any isoform synthesized with heavy-isotopes of carbon and/or nitrogen to produce Tau protein with a higher molecular mass for the surrogate Tau peptide than unlabeled Tau.

As used herein, the term “Tau Calibration Standard” refers to purified recombinant Tau of any isoform of a known concentration that is used as a reference for the quantification of Tau in samples with unknown levels.

As used herein, the term “MRM Transition Pair” refers to a product and precursor ion pair that is produced in the mass spectrometer and monitored for abundance in a sample.

As used herein, the term “product ion transitions” refers to the ion species produced following ion fragmentation in the mass spectrometer.

As used herein, “biomarker” refers to a measurable biological characteristic that reflects a physiological, pharmacological or disease process in animals or humans.

As used herein, an “analyte” is a molecule or peptide that is measured by mass spectrometry.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989). The variable regions of each light/heavy chain pair form the antibody binding site. Thus, in general, an intact antibody has two binding sites. With the exception of bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat et al. (1977) J. Biol. Chem. 252:6609-6616; Chothia et al. (1987) J. Mol. Biol. 196:901-917 or Chothia et al. (1989) Nature 342:878-883.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The term “sequence identity” refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

The following references relate to BLAST algorithms often used for sequence analysis: BLAST ALGORITHMS: Altschul et al. (1990) J. Mol. Biol. 215:403-410; Gish et al. (1993) Nature Genet. 3:266-272; Madden et al. (1996) Meth. Enzymol. 266:131-141; Altschul et al. (1997) Nucleic Acids Res. 25:3389-3402; Zhang et al. (1997) Genome Res. 7:649-656; Wootton et al. (1993) Comput. Chem. 17:149-163; Hancock et al. (1994) Comput. Appl. Biosci. 10:67-70; ALIGNMENT SCORING SYSTEMS: Dayhoff et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz et al. “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul (1991) J. Mol. Biol. 219:555-565; States et al. (1991) Methods 3:66-70; Henikoff et al. (1992) Proc. Natl. Acad. Sci. USA 89:10915-10919; Altschul et al. (1993) J. Mol. Evol. 36:290-300; ALIGNMENT STATISTICS: Karlin et al. (1990) Proc. Natl. Acad. Sci. USA 87:2264-2268; Karlin et al. (1993) Proc. Natl. Acad. Sci. USA 90:5873-5877; Dembo et al. (1994) Ann. Prob. 22:2022-2039; and Altschul “Evaluating the statistical significance of multiple distinct local alignments.” in Theoretical and Computational Methods in Genome Research (S. Suhai, ed.), (1997) pp. 1-14, Plenum, N.Y.

### IA MS/MS Assay

Immunoassay methods are particularly popular for the detection of proteins in biological fluids. However, immunoassays are often unable to distinguish between chemically and structurally similar peptides which may differ by only a small change outside of the region recognized by the capture and detection antibodies. In addition, immunoassays are often characterized by a limited linear dynamic range for quantification.

Mass spectrometry-based assays are widely used in the field of proteomics and hold promise for the qualification of biomarkers and the development of in vitro quantitation assays suitable for drug development and clinical and diagnostic applications. While both immunoassays, and MS-based assays can detect and determine protein concentration, mass spectrometric detection is generally accepted to have greater selectivity, which may enable the identification of mass-distinctive protein variants. The occurrence of protein variants during a pathological process may play a significant role in an ongoing disease process. Immunoaffinity enriched and trypsin digested Tau from a biological sample can be used in two ways. First, peptides specific to Tau variants can be identified by profiling using a high-resolution mass spectrometer and data analysis using software designed for feature extraction and database searching. For the quantification of specific peptide variants, LC-MS/MS analysis is utilized.

Innovations in separation science and mass spectroscopy have given rise to instruments and methods that are capable of high-throughput automated assay protocols. As shown herein mass spectrometry provides an ideal platform for the development of a quantitative assay for total-Tau and detection of protein variants in a highly selective bioanalytical assay, which may provide significant insights in the biomarker and/or diagnostic fields.

The IA MS/MS bioanalytical total Tau assay disclosed herein employs a surrogate tryptic peptide that is present in all isoforms of Tau. As disclosed herein the surrogate peptide used to exemplify the invention herein was selected because it is not (or is only minimally likely) to undergo post-translational modification.

Developing a mass spectrometry assay suitable for clinical sample analysis requires meeting key requirements for assay precision and robustness. The use of heavy isotope labeled Tau protein as an internal standard accounts for variability throughout sample processing, as it is added at the very beginning of the procedure before immunoaffinity enrichment and trypsin digestion. For detection, the LC-MS/MS Trizaic nanoTile system was found to be sufficiently robust for clinical analysis, with fast cycle times, reproducible peptide retention, times and no need for user intervention during the course of hundreds of sample analyses.

The assays provided herein represent an advancement over prior assays, such as those described by Portelius et al. (2008) J. Proteome Res. 7:2114, as they exhibit increased sensitivity that now permits the quantitation of total CSF Tau levels. The increase in sensitivity is likely due to the more sensitive analytical mass spectrometers used in our study. The use of an heavy-isotope labeled internal standard was necessary to decrease variability by enabling correction for differences between samples during sample processing. Prior art methods may employ a perchloric acid precipitation step prior to immunoprecipitation that was not necessary in our method due to the effectiveness of the optimized immunoprecipitation procedure.

As shown herein the disclosed invention provides an assay for quantification of Tau in cerebrospinal fluid using immunoaffinity enrichment of target analyte prior to tryptic digestion and mass spectrometry detection of an analyte-specific surrogate peptide. The sensitivity of the assay, when combined with the specificity of mass spectrometry, can be used to understand modifications or changes in the Tau protein that are relatively uncommon but may have a profound relationship with disease progression. One of skill in the art will readily acknowledge that MRM analysis can easily be developed to monitor multiple Tau-derived peptides, including those containing post-translational modifications.

### Tau as a Biomarker for AD

A biomarker can be simply defined as a measurable biological characteristic that can serve as an indicator of normal or pathogenic processes, or as a tool to monitor pharmacological responses to therapeutic drugs. The concentration of the microtubule binding protein Tau present in CSF is a common clinical biomarker for AD. Due to the low abundance of Tau in CSF, measurements of Tau are typically performed using immunoassays. In practice, Tau is a very difficult protein to quantitate, partially because it is present in low abundance in CSF. In healthy individuals the concentration of Tau in the CSF can range from 200-400 pg/ml (5-10 pM). In AD patients the concentration is typically elevated and can range from 400-800 pg/ml (10-20 pM). A useful clinical assay should have a LOQ below the range found in subjects from normal or diseased populations, whichever is lower. As discussed in Example 5, one assay of the present invention has an LLOQ of 0.25 pM, well below the Tau levels observed in CSF from healthy individuals and AD patients.

In addition, there are 6 distinct isoforms of Tau (352, 381, 383, 410, 412, 441) and an unknown number of potential degradation products circulating in CSF, many containing a variety of post-translational modifications (PTMs) including phosphorylations, glycosylations, acetylations and others. Thus, specificity is of particular importance for the analytical quantification of Tau in CSF.

In the case of CSF total-Tau, multiple immunoassays exist that rely on different combinations of capture and detection antibodies with uncertain specificity. Two commonly used commercial Tau assay kits are the Innotest hTau from Fujirebio (Malvern, Pa. USA) (formerly Innogenetics) and the Human Total Tau Kit from Meso Scale Discovery (MSD, Rockville, Md., USA). The Innotest Htau is a solid-phase colorimetric ELISA that utilizes monoclonal Ab AT120 as a capture and both monoclonal Abs HT7 and BT2 for detection. The Meso Scale Human Tau is a solid-phase electrochemiluminescense ELISA that uses two monoclonal antibodies for capture and detection. At the present time, no standardized reference material or reference method has been identified that can be used to reconcile differences between assays results. Kang et al. (2013) Clin. Chem. 59:903-16. In contrast to immunoassays, quantitative LC-MS/MS methods directly measure the target peptide with specificity based on the peptide's retention time, mass/charge and fragmentation. Becker & Hoofnagle (2012) Bioanalysis 4:281-90; Hoofnagle & Wener (2009) J. Immunol. Methods 347:3-11; Lassman et al. (2012) Rapid Commun. Mass Spectrom. 26:101-8.

As an alternative to immunoassays, investigators have published reports evaluating the use of tryptic digestion, followed by analyte-specific peptide detection and quantification of LC-MS/MS. In contrast to immunoassays, quantitative LC-MS/MS methods directly detect the target peptide with a high degree of specificity based on the peptide's retention time, mass/charge and fragmentation. Anderson & Hunter (2006) Mol. Cell. Proteomics 5:573-88; Domanski et al. (2012) Proteomics 12:1222-43; Ong & Mann (2005) Nat. Chem. Biol. 1:252-62. However, generally speaking LC-MS/MS assays without analyte enrichment lack the requisite sensitivity for the detection and direct quantification of low-abundance proteins, such as Tau in human biological fluids.

The SISCAPA (stable isotope standards and capture by anti-peptide antibodies) is an alternative method to quantitate biomarkers present in biological fluids. This method involves the enzymatic digestion of all sample protein with trypsin before immunoprecipitation with specific antibodies for the surrogate peptide analyte. Stable isotope-labeled internal standard peptides are added to account for differences in recovery between samples that occur during the sample preparation procedure, but do not account for potential differences in trypsin digestion efficiency between samples. Because only the surrogate peptide analyte is immunoaffinity purified, no information can be obtained regarding different forms (e.g. isoforms or phosphorylated forms) of the protein containing the surrogate peptide, particularly those forms with modifications outside the surrogate peptide region. Conversely, immunoprecipitation of the full-length protein analyte before trypsin digestion provides the opportunity for additional analyses of multiple features, variants or modifications of the protein that was immunoaffinity enriched. For example, additional surrogate peptides specific to a particular protein isoform could also be measured in the sample to provide information about isoform abundance. Therefore, it is useful to immunoprecipitate the intact protein before digestion when it is desired to monitor multiple features on the protein.

### Internal Tau Standard

Developing a mass spectrometry assay suitable for clinical sample analysis requires meeting key requirements for assay precision and robustness. The use of heavy isotope labeled Tau protein as an internal standard accounts for variability throughout sample processing, as it is added at the very beginning of the procedure before immunoaffinity enrichment. Extensive up-front testing of the internal standard ensured the Tau internal standard protein was recovered equally with the endogenous Tau.

The isotope-labeled internal Tau standard described herein is a recombinant Tau protein having the same amino acid sequence as an endogenous Tau isoform with a heavy isotope label incorporated into at least one amino acid residue. Generally speaking the labeled amino acid residues that are incorporated should increase the mass of the peptide without affecting its chemical properties, and the mass shift resulting from the presence of the isotope labels must be sufficient to allow the MS/MS to distinguish the internal standard (IS) from endogenous Tau analyte signals. As shown herein suitable heavy isotope labels include, but are not limited to ²H, ¹³C, and ¹⁵N. The 412 isoform of Tau was used as IS in the experiments described herein, although experiments demonstrated no bias in immunocapture affinity among isoforms, suggesting that isoform should not matter. IS was used at 2 pM final concentration in a 1 mL sample, which was similar to endogenous levels and sufficient to provide a strong and reproducible response.

### Selection of Total Tau Surrogate Peptide

The MS/MS analysis of intact polypeptides above 10 kDa typically fails to achieve sufficient selectivity and sensitivity due to issues with multiple charging and wide isotopic distribution. In practice, these issues are typically addressed by using proteases to digest the target protein to produce smaller surrogate peptides, which can be analyzed in place of the intact target protein and which allow for lower limits of quantification to be achieved. Published reports have established that surrogate peptides can be successfully used for accurate protein quantification, and indicate that up to a 250-fold improvement in LLOQ.

An optimal surrogate peptide is identified empirically as the peptide providing the highest assay sensitivity with the lowest assay variability, starting from a list of potential peptides as determined in silico. Additional factors include peptide size and probability of phosphorylation or other variation. The mass spectrometry analysis method settings are optimized using a synthetic version of each potential surrogate peptide, which can be produced in high quantities. Optimization includes identification of the most intense fragment ion and collision energy for use as the MRM transition.

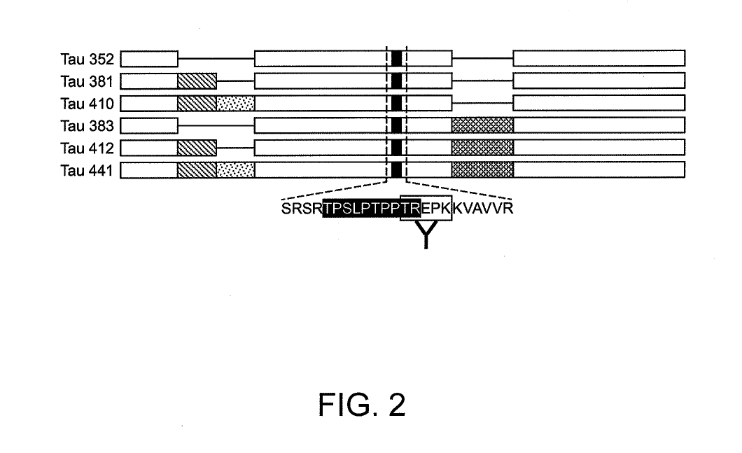

In order to identify candidate Tau surrogate peptides suitable for use in IA-MS assay, A blast homology comparison between the six Tau isoforms (352, 381, 383, 410, 412, 441) was performed to identify conserved regions among the isoforms (FIG. 2). An in silico tryptic digest revealed multiple peptides amenable to MS analysis that are conserved between the six isoforms of human Tau. Starting with the sequences for all Tau isoforms, regions of consensus were identified. A list of tryptic peptides originating from these shared sequences was then assembled and theoretical masses were calculated. Such analysis can be performed manually, or through use of software, such Skyline from the MacCoss Lab, University of Washington. Many peptides could be excluded for being too short (less than 7 amino acids) or containing a known modification site. Remaining peptides were then evaluated using synthetic peptides for each. The optimal transition settings (parent ion and fragment ion mass/charge settings) for each peptide were determined by a “tuning” process. This involves infusion of the peptides into the mass spectrometer to determine the optimal mass setting for the peptide, and to find the optimal collision energy that will produce a prominent fragment ion. Following tuning for each peptide, they were tested side by side for performance in the assay.

The optimal surrogate peptide was selected based on factors including signal intensity, the background level, the resolution of the peak from noise, and the chromatographic performance. See Examples 1 and 4.

The selected surrogate peptide, TPSLPTPPTR (SEQ ID NO:1), can in principle be used to detect Tau not only in human samples, but also in mouse models expressing human Tau, and also in non-human primate (NHP) studies since antibody 13A6 was found to immunoaffinity enrich rhesus macaque: (Macaca mulatta) Tau protein despite a one amino acid difference in the analogous surrogate peptide sequence (data not shown).

### Internal Standard Tau Peptide

An advantage of using intact recombinant Tau as an Internal Standard (IS), instead of a proteolytic peptide, is that the use of the intact target proteins allows internal standardization to commence at the start of sample preparation and therefore accounts for variations of both immunoaffinity enrichment and digestion. Heavy isotope labeled standards are useful tools for mass spectrometry based assays as these standards have the identical chemical properties as the endogenous protein but are analyzed separately using a different mass/charge setting. If added to the sample as early as possible, they can act to correct for any variation throughout the entire sample preparation procedure, thereby providing much more precise measurements. To take full advantage of the power of this approach, an intact (full-length) IS protein was required so it would contain both the antibody epitope required for immunoaffinity enrichment as well as all potential surrogate peptides for total Tau quantitation. A shorter Tau peptide IS containing these two elements alone was also avoided since it may perform slightly differently than the full length protein in terms of antibody binding affinity or digestion efficiency.

The suitability of using this single isoform recombinant protein as an internal standard for all endogenous isoforms was tested to ensure there was no bias in immunoaffinity enrichment. Testing consisted of mixing internal standard Tau with unlabeled Tau at various concentrations in buffer and measuring the labeled to unlabeled ratio before and after immunoaffinity enrichment. No difference in the ratio was observed with IA enrichment, indicating that no bias was present. Extensive up-front testing of the internal standard ensured the labeled Tau internal standard protein was recovered equally with the endogenous Tau.

### Identification and Characterization of Anti-Tau Antibody

Development of any antibody-based immunological assay begins with the identification of an antibody capable of enriching the target analyte from a biological sample. It is well known that antibody-directed analyte enrichment rapidly reduces sample complexity with a high degree of specificity. The use of an immunoaffinity enrichment step, which relies on the specificity of a monoclonal antibody to capture polypeptides comprising a specific epitope, can in practice simplify and enrich an analyte from a biological sample by a factor of more than 10-, 50- or even 100-fold. In theory, immunoaffinity capture of Tau could result in greater than 100-fold enrichment, whereas prior art methods employing less selective methods, like SPE or perchloric acid precipitation will retain all proteins similar to Tau and therefore may only enrich Tau in the sample by 10 to 100-fold at best.

The subsequent introduction of the enriched sample into a LC-MS/MS instrument provides the analytical step with sufficient concentration and separation space to discriminate among distinct isoforms and/or species of the analyte comprising distinct structural features (e.g., variations and/or post-translational modifications).

In order to identify a suitable antibody for use in the immunoaffinity steps of the disclosed assay, polyclonal rabbit antisera and murine hybridomas producing monoclonal anti-Tau antibodies were both raised against an 166 amino acid Tau fragment containing the central region of the human Tau protein (which is shared among all six of the Tau isoforms).

A single monoclonal clone developed in-house (clone 13A6) was selected for use in this assay due to availability and good assay performance. To ensure that clone 13A6 equally enriches all Tau isoforms without bias, recoveries of each recombinant isoform of Tau were tested and compared. Samples of recombinant Tau (approximately 100 pM) for each isoform were quantified after trypsin digestion with or without using immunoaffinity purification with 13A6. For each isoform, the percent recovery was calculated. Recoveries for all six isoforms ranged from 60% to 75% and did not reveal any bias among isoforms (data not shown).

The present invention provides an isolated antibody or antigen binding fragment thereof that specifically binds Tau and comprises the V_{L} domain of SEQ ID NO: 39 and the VH domain of SEQ ID NO: 37. The present invention also provides an isolated antibody, or antigen binding fragment thereof, that specifically binds Tau and comprises either the V_{H} domain of SEQ ID NO: 37 or the VL domain of an antibody that comprises the VL domain of SEQ ID NO: 39.

In alternative embodiments, an isolated antibody, or antigen binding fragment thereof, that is disclosed herein (and used to exemplify the bioanalytic assay of the invention), binds Tau can comprise one, two, three, four, five, or six of the complementarity determining regions (CDRs) of the antibody disclosed herein (e.g., Table 2, Table 3)

TABLE 2
Light Chain CDRs and VL sequences

| Antibody | CDR1 | CDR2 | CDR3 | VL |
|---|---|---|---|---|
| 13A6 | SEQ ID | SEQ ID | SEQ ID | SEQ ID |
| | NO: 43 | NO: 44 | NO: 45 | NO: 39 |

TABLE 3
Heavy Chain CDRs and VH sequences

| Antibody | CDR1 | CDR2 | CDR3 | VH |
|---|---|---|---|---|
| 13A6 | SEQ ID | SEQ ID | SEQ ID | SEQ ID |
| | NO: 40 | NO: 41 | NO: 42 | NO: 37 |

The present invention further provides an isolated antibody or antigen-binding fragment thereof that binds Tau comprising at least one antibody light chain variable (V_{L}) domain comprising one or more of CDR-L1, CDR-L2 and CDR-L3 of 13A6. The present invention also provides isolated antibody or antigen-binding fragment thereof that binds Tau comprising at least one antibody heavy chain variable (VH) domain comprising one or more of CDR-H1, CDR-H2 or CDR-H3 of 13A6.

### Epitope Binding

The present invention further provides antibodies or antigen binding fragments thereof that bind to the same epitope on human Tau as antibody 13A6. Epitope mapping performed on the 13A6 mAb revealed binding of a peptide with sequence TREPK (Tau amino acids 220-224, SEQ ID NO:5). See Example 2. As shown in FIG. 2, the amino acids “TR” of this epitope are also contained within the peptide selected for MRM analysis: TPSLPTPPTR (SEQ ID NO:1).

In another embodiment, antibodies that are able to cross-block binding of any of the antibodies disclosed herein are provided. A first antibody is considered to cross-block binding of a second antibody if rebinding the target with the first antibody to saturation increases the concentration of second antibody needed to achieve half-maximal binding of the target by 2-, 3-, 4-, 5-, 10-, 20-, 50-, 100-, 200-fold or more.

### Binding Affinity

By way of example, and not limitation, the antibodies disclosed herein may bind human Tau with a K_{D} value of at least about 100 nM (1×10⁻⁷ M); at least about 10 nM; or at least about 1 nM. Antibody 13A6 of the present invention binds to human Tau with K_{D} of 9.7±6.1 nM.

A polyclonal antibody is an antibody which was produced among or in the presence of one or more other, non-identical antibodies. In general, polyclonal antibodies are produced from collections of different B-lymphocytes, e.g. the B-lymphocyte of an animal treated with an immunogen of interest, which produces a population of different antibodies but which are all directed to the immunogen. Usually, polyclonal antibodies are obtained directly from an immunized animal, e.g. spleen, serum or ascites fluid.

Hybridoma cells that produce parental (e.g. rodent) monoclonal anti-Tau antibodies may be produced by methods which are commonly known in the art. These methods include, but are not limited to, the hybridoma technique originally developed by Kohler et al. (1975) (Nature 256:495-497), as well as the trioma technique (Hering et al. (1988) Biomed. Biochim. Acta. 47:211-216 and Hagiwara et al. (1993) Hum. Antibod. Hybridomas 4:15), the human B-cell hybridoma technique (Kozbor et al. (1983) Immunology Today 4:72 and Cote et al. (1983) Proc. Natl. Acad. Sci. U.S.A 80:2026-2030), the EBV-hybridoma technique (Cole et al., in Monoclonal Antibodies and Cancer Therapy, Alan R. Liss, Inc., pp. 77-96, 1985), and electric field based electrofusion using a Cyto Pulse large chamber cull fusion electroporator (Cyto Pulse Sciences, Inc., Glen Burnie, Md., USA). Preferably, mouse splenocytes are isolated and fused with PEG or by electrofusion to a mouse myeloma cell line based upon standard protocols. The resulting hybridomas may then be screened for the production of antigen-specific antibodies. For example, single cell suspensions of splenic lymphocytes from immunized mice may by fused to one-sixth the number of P3X63-Ag8.653 nonsecreting mouse myeloma cells (ATCC, CRL 1580) with 50% PEG. Cells may be plated at approximately 2×10⁵ cells/mL in a flat bottom microtiter plate, followed by a two week incubation in selective medium containing 20% fetal Clone Serum, 18% “653” conditioned media, 5% origen (IGEN), 4 mM L-glutamine, 1 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1× HAT (Sigma; the HAT is added 24 hours after the fusion). After two weeks, cells may be cultured in medium in which the HAT is replaced with HT. Individual wells may then be screened by ELISA for anti-Tau monoclonal IgG antibodies. Once extensive hybridoma growth occurs, medium can be observed usually after 10-14 days. The antibody secreting hybridomas may be replated, screened again, and if still positive for human IgG, anti-Tau monoclonal antibodies, can be subcloned at least twice by limiting dilution. The stable subclones may then be cultured in vitro to generate small amounts of antibody in tissue culture medium for characterization.

The anti-Tau antibodies disclosed herein may also be produced recombinantly (e.g., in an E. coli/T7 expression system as discussed above). In this embodiment, nucleic acids encoding the antibody molecules of the invention (e.g., VH or VL) may be inserted into a pET-based plasmid and expressed in the E. coli/T7 system. There are several methods by which to produce recombinant antibodies which are known in the art. One example of a method for recombinant production of antibodies is disclosed in U.S. Pat. No. 4,816,567. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455. Anti-Tau antibodies can also be synthesized by any of the methods set forth in U.S. Pat. No. 6,331,415.

Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and 0 338 841.

### Optimization of Immunoaffinity Enrichment Steps

The antibody used to capture and enrich the Tau protein analyte from the biological sample is covalently bound to a solid phase particle to facilitate the incorporation of a washing step into the immunoaffinity portion of the assay. The immunoaffinity process is a one-step process in which anti-human Tau antibody covalently bound to a solid phase particle is incubated with a CSF sample comprising endogenous Tau analyte and an internal standard which is a stable isotope labeled recombinant Tau isoform.

Affinity purified rabbit polyclonal antibodies to human Tau and multiple mouse monoclonal antibodies (commercially available and produced in-house) were coupled to tosyl-activated magnetic beads and evaluated for use in the immunoselection step of the invention. Polyclonal antibodies were found to result in higher background signal compared to monoclonals, with the majority of monoclonal antibodies tested performing well, but with reproducible differences in background, likely due to differences in specificity.

Following antibody selection, multiple parameters of the Tau IA binding, wash, elution, and digestion steps were optimized. The quantity of beads required for optimal Tau recovery was determined to be at least 0.05 mg beads; the trypsin digestion was found to be optimal using 0.5 μg trypsin per sample overnight.

As shown herein paramagnetic beads provide a suitable solid phase particle, however one of skill in the art will readily acknowledge that alternative solid phase particles such as polymer beads or commercially available surface-activated beads can be used to immobilize the anti-Tau antibody. The final choice of particle and coupling chemistry used to covalently bind the antibody to the particle will depend upon the specificity and affinity of the anti-Tau antibody and the details of the immunoaffinity and digestion steps of the protocol. For example, nonmagnetic beads could be used as the solid phase support in methods of the present invention, as well as a perfusable solid support (such as MSIA tips), or antibody could be coated on a solid surface (plates).

### LC-MS/NIS Parameters

For detection, the LC-MS/MS Trizaic nanoTile system was found to be sufficiently robust for clinical analysis, with fast cycle times, reproducible peptide retention times and no need for user intervention during the course of hundreds of sample analyses.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

The present invention is not to be limited in scope by the specific embodiments described herein; the embodiments specifically set forth herein are not necessarily intended to be exhaustive. Indeed, various modifications of the invention in addition to those described herein will become apparent to those skilled in the art from the foregoing description and the accompanying figures. Such modifications are intended to fall within the scope of the appended claims.

### EXAMPLES

The following examples are intended to exemplify the present invention and not to be a limitation thereof. The methods and compositions disclosed below fall within the scope of the present invention.

### General Materials and Methods

### Reagents

Trypsin Gold Mass Spec Grade was purchased from Promega (Madison, Wis., USA). Phosphate buffered saline (PBS) was purchased from Sigma-Aldrich (St Louis, Mo., USA). Synthetic peptides corresponding to the tryptic surrogate total-Tau analytes were produced at New England Peptide, Inc (Gardner, Mass., USA).

### Anti-Tau Monoclonal Antibody

Mouse monoclonal and rabbit affinity purified polyclonal anti-Tau antibodies were both raised against a 166 amino acid Tau fragment containing the central region of the protein that is shared among Tau isoforms. One resulting clone (13A6) was chosen for use in the immunoaffinity enrichment of Tau.

### Preparation of Antibody Coupled Beads

13A6 anti-Tau antibody was covalently coupled to MyOne Tosylactivated DYNABEADS (Life Technologies, Grand Island, N.Y., USA) following the manufacturer's recommended protocol. For enough material to process approximately 250 samples, 2 mg antibodies were coupled to 50 mg beads in a conjugation batch and diluted to 2.5 ml (20 mg/ml) until use.

### Equipment

Magnetic Particle Concentrator
Fisher Vortex Genie 2 (cat #12-812)
37° C. incubator with tube rotator
Benchtop centrifuge

### Materials

monoclonal antibody clone 13A6 (1 mg/ml in PBS)
DYNABEADS MyOne Tosylactivated (Invitrogen cat#655.02D)
Sodium Tetraborate Decahydrate (Na2B4O7.10H2O; Sigma #S9640)
Sodium Hydroxide 5N, (EMD cat#SX0607L/3)
Ammonium Sulfate (Fisher cat#A7013)
Phosphate Buffered Saline (PBS; Hyclone cat#SH30256.02)
Tween-20 (Bio-Rad cat#170-6531)
Sodium Azide (NaN3; Fisher cat#S2271), 2% stock solution in diH2O
Bovine Serum Albumin, 2 mg/ml (BSA; Pierce)
Amicon Ultra-15 centrifugal Filter Units 10K MWCO (Millipore #UFC901024)

### Preparation of Working Solutions

Borate coating buffer, 0.1M Sodium Borate pH 9.5 Prepare by dissolving 19 g Sodium Tetraborate Decahydrate in 500 ml diH2O and adjusting the pH to 9.5 with dropwise 5N Sodium Hydroxide. Good for 6 months stored at RT. Check pH before each use.

3M Ammonium Sulfate solution Weigh out 1.98 g solid Ammonium Sulfate and transfer to a graduated conical tube. Add Borate coating buffer to a total volume of 5 ml. Vortex well to dissolve. Prepare fresh as needed.

Bead Storage Buffer (PBS+0.1% Bovine Serum Albumin) Add 25 μl Tween-20 to 50 ml PBS in a 50 ml conical tube. Mix gently and store at RT for up to 1 month at 4° C.

Preparation of 2.5 ml (20 mg/ml) Coupled DYNABEADS

1. Vortex stock vial of 10 ml MyOne Tosylactivated Dynabeads 30 sec to resuspend.

2. Transfer 500 μl beads to a 2 ml polypropylene tube. Place the tube in the magnetic stand and wait 2 min to separate beads. Aspirate clear bead storage buffer.

3. Wash beads with 1 ml of borate coating buffer, vortexing 1 min, and placing tube back in magnetic stand for 2 min. During binding, gently invert stand to remove any beads from the cap. Aspirate clear wash buffer leaving the washed beads in the tube.

4. Repeat wash step with 1 ml borate coating buffer wash for a total of two washes.

5. Add 2 ml borate coupling buffer to 2 ml (2 mg) of a 1 mg/ml mAb solution. Use Amicon concentrator to concentrate solution to approximately 1 ml. Add an additional 3 ml borate coating buffer to concentrator and repeat concentration to approximately 0.7 ml. Add enough borate coating buffer to yield a 735 μl solution. Transfer concentrated antibodies to eppendorf tube containing washed bead pellet.

6. Add 415 μl 3M ammonium sulfate to coupling reaction. Vortex well.

7. Incubate coupling reaction on rotator at 37° C. for 16-24 hr.

8. The next day, collect beads by placing the reaction tube in the magnetic stand and transfer clear liquid to new eppendorf tube.

9. Resuspend beads in 1 ml PBS+0.5% BSA+0.05% Tween-20 and rotate overnight at 37° C.

10. The next day, wash beads three times with 1 ml Bead Storage Buffer using magnetic stand to collect beads. After the final wash, resuspend beads in a total of 2.5 ml Bead Storage Buffer+0.02% NaN3.

11. Store coupled beads at 4° C. for up to 1 year.

### Tau Reference Materials

A recombinant Tau calibration standard (Tau 381AA isoform) was produced by Proteos and quantitated by amino acid analysis (AAA Service Laboratory, Inc.). A larger full length ¹³C-¹⁵N Arg/Lys labeled Tau (412AA) internal standard protein (IS) was produced by Promise Proteomics (Grenoble, France). A panel of six unlabeled recombinant Tau isoforms was obtained from rPeptide (Bogart, Ga., USA). 10 nM stock solutions of the recombinant proteins in PBS+0.2% BSA and 1 nM IS in PBS+0.2% BSA were prepared and frozen in single use aliquots and stored at −80° C.

### Calibration and QC Samples

For each analysis, an eight-point calibration curve was prepared daily from 50 pM to 0.781 pM using a two-fold serial dilution of recombinant Tau381 in PBS. QC samples were prepared using different ratios of commercially available remnant healthy CSF and post-mortem human CSF both from Bioreclamation (Hicksville, N.Y., USA) at three different levels, prepared and frozen in single use aliquots and stored at −80 C. A set of each of the three QC samples were included in each analytical run.

### Immunoaffinity Enrichment and Protein Digestion

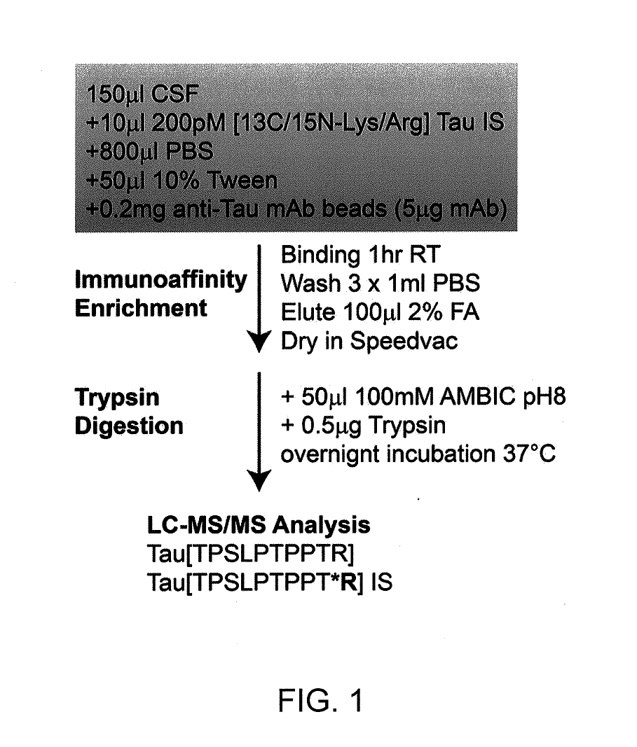

The workflow for the optimized IA-MS analysis of Tau in CSF is shown in FIG. 1. CSF samples, calibration standards, and QC samples were added to wells of a 1.8 ml deepwell plate and diluted (when applicable) to 1 ml using PBS.

To each sample, the following were added: 10 μl 200 pM Tau412 IS, 50 μl 10% Tween-20, and 10 μl suspension (0.2 mg) of anti-Tau mAb coated magnetic beads. The plate was sealed and mixed at RT for 1 hr at 1200 rpm. Magnetic beads were washed 3 times with 1 ml PBS with the aid of a plate stand with magnetic posts that pull the magnetic beads to the side of each well to allow for liquid aspiration without noticeable bead loss.

Proteins were eluted from the washed beads in 100 μl 2% Formic Acid with constant shaking at 1200 rpm at RT for 20 min, transferred to a new 96-well plate, and evaporated to dryness in a speed-vac. Dried proteins were resuspended in a mixture of 0.5 μg trypsin in 50 μl 100 mM pH8.0 Ammonium Bicarbonate and digested overnight (15-18H) with mixing at 1200 rpm at 37 C.

### LC-MS/MS Quantitation of Total-Tau in CSF

A Waters nanoAcuity system (Milford, Mass., USA) was used to load 8 μl of each digested sample onto an 85 μm Trizaic nanotile containing a trapping column at 4 μl/min for 5 min in 100% Buffer A (0.1% Formic Acid in Water). Elution of bound proteins and chromatography was performed with a linear gradient from 5-50% Buffer B (0.1% Formic Acid in Acetonitrile) over 4 min followed by a 1 min 50-90% gradient with a constant tile temperature of 65° C. The column wash was held at 90% Buffer B for 1.5 min then equilibrated at 10% Buffer B for 1.5 min.

Mass spectrometry was performed using a Waters Xevo-TQS mass spectrometer (Milford, Mass., USA). The transitions of unlabeled and heavy isotope labeled internal standard peptides used in MRM assays are listed in Table 1. Instrument-specific parameters for collision energy were experimentally determined during infusion of synthetic peptides. A capillary voltage of 3.0 kV was used for all analyses. Resolution settings were optimized for signal/noise and were set as follows: LM 1 resolution=2.5, HM 1 resolution=15.1, Ion energy 1=0.2, LM 2 resolution=2.8, HM 2 resolution=15.5, Ion energy 2=0.8.

### LC-MS Profiling for the Characterization of Post-Translational Modifications

Samples were analyzed by a reverse phase nano-UPLC (Waters nanoAcquity) coupled to a Velos Orbitrap hybrid mass spectrometer with electron transfer dissociation (ThermoFisher). 1 μL of digested sample was loaded onto a capillary sample trap column (100 um ID, 2.5 cm; ProteoPrep 2; IntegraFrit; New Objectives) and desalted on line for 5 min at 3 μL/min with 99.5% solvent A [100% HPLC grade water, 0.1% formic acid] 0.5% solvent B [100% HPLC grade acetonitrile, 0.1% formic acid]. After 5 minutes the flow rate was reduced to 0.25 μL/min and peptides were eluted into packed spray tip column (75 μm i.d., 190 μm o.d.×10 cm; Reprosil-Pur C18; PicoFrit 15 μm; New Objectives). Bound peptides were eluted from the column via a 75 min solvent gradient program.

High resolution mass spectra were acquired at a rate of 1 spectrum per second. 15 data-dependent MS/MS spectra were acquired for the most intense ions in each full scan spectra to provide amino acid sequence information for selected peptide ions.

Acquired MS data were uploaded to the Elucidator data analysis system (Rosetta Biosoftware, version 4.0) as previously described (Andersen et al. (2010) Sci. Transl. Med. 2:43ra55; Paweletz et al. (2010) J. Proteome Res. 9:1392-401) for feature extraction, quantitative analysis and searched against an internal database containing human sequences obtained from National Center for Biotechnology Information (NCBI) concatenated with their reverse counterparts using Mascot. Parameters for the searches included the following: Trypsin enzyme specificity; static modifications of 57.02 Da on Cys (Carbamidomethylation); differential modification of 79.9 Da on Ser, Thr, and Tyr (phosphorylation) and 15.99 Da on Met (oxidation). The precursor ion mass tolerance was set as 50 ppm and the fragment-ion mass tolerance was set as +/−0.8 Da Mono/Mono. Each MS/MS spectra exhibiting possible phosphorylation were manually validated. Peptide intensities were derived based on areas under the ion chromatographic curves as determined by Elucidator.

### Fit-for-Purpose Analytical Validation

Intraday reproducibility was determined by measuring the concentration of three QC samples from six different single-use vials prepared and analyzed on a single plate. Inter-day reproducibility was determined by measuring the concentration of the three QC samples from different single-use vials prepared on six different plates and prepared and analyzed on six different days. Dilution linearity was determined by diluting commercially available CSF in PBS, maintaining a final volume 1 mL. Spike recovery was performed on a single lot of CSF spiked with 1.1, 4.0 and 11.1 pM recombinant Tau before immunoaffinity precipitation.

CSF Samples from Healthy and Alzheimer's Disease (AD) Donors

A panel of age matched CSF from normal cognition donors (n=50) and Alzheimer's Disease diagnosed donors (n=77) were obtained from PrecisionMed Inc (Solana Beach, Calif., USA). Tau levels in CSF were evaluated by immunoassay with the Human Total Tau kit (K151LAE) from MSD (Rockville, Md., USA), following the manufacturing instructions. Statistical analysis (t-test) and plotting of the data was performed using GraphPad Prism 5.

### Example 1

### Selection of Total Tau Surrogate Peptide

Purpose:

To identify candidate Tau surrogate peptides suitable for use in JA-MS assay.

A blast homology comparison between the six Tau isoforms (352, 381, 383, 410, 412, 441) was performed to identify conserved regions among the isoforms (FIG. 2). An in silico tryptic digest revealed multiple peptides amenable to MS analysis that are conserved between the six isoforms of human Tau. Of these, three peptides were selected as potential total-Tau surrogate peptides TPSLPTPPTR (SEQ ID NO: 1); LQTAPVPMPDLK (SEQ ID NO: 2); IGSLDNITHVPGGGNK (SEQ ID NO:3) based on a number of factors including sequence uniqueness, reduced likelihood of PTMs, and amenability to LC-MS analysis.

For each of these peptides, two product ion transitions were selected so that specificity could be confirmed. Table 4 provides a list of selected Tau derived peptides and associated PTMs identified by data dependent analysis of immunoaffinity enriched post mortem CSF.

TABLE 4
Tau Peptide Post-Translational Modification
Levels

| | | | | Relative % |
|---|---|---|---|---|
| | | | | PTM (PTM |
| | SEQ | | | intensity/ |
| | ID | Mascot | Peak | combined |
| Peptide Sequence | NO | Score | intensity | intensity) |
| TPSLPTPPTR | 1 | 44.55 | 8.7E+06 | 1.7% |
| TPS(Phos)LPTPPTR | 9 | 20.83 | 1.5E+05 | |
| STPTAEDVTAPLVDEGAPGK | 13 | 71.57 | 2.3E+04 | 53% |
| ST(Phos) | 14 | 50.61 | 2.6E+04 | |
| PTAEDVTAPLVDEGAPGK | | | | |
| SPVVSGDTSPR | 17 | 32.35 | 7.5E+04 | 39% |
| SPVVSGDTS(Phos)PR | 18 | 60.35 | 4.7E+04 | |
| LQTAPVPMPDLK | 2 | 64.24 | 4.1E+06 | 25% |
| LQTAPVPM(Ox)PDLK | 23 | 46.58 | 1.4E+06 | |

Results:

Analysis of a tryptic digest of recombinant Tau381 as well as an equimolar mix of synthetic versions of the three monitored Tau peptides revealed that the peptide TPSLPTPPTR provided the most intense MRM signal intensity and the primary transition for this peptide was selected as the primary surrogate peptide for total-Tau throughout method development and validation.

### Example 2

### Identification and Characterization of Anti-Human Tau Antibody

Mouse monoclonal and rabbit affinity purified polyclonal anti-Tau antibodies were both raised against a 166 amino acid Tau fragment containing the central region of the protein that is shared among Tau isoforms. This 166 amino acid fragment corresponds to amino acids 104-259 of human Tau 441 isoform disclosed at NP_{—}005901.2. Sequences for selected mouse monoclonal antibody 13A6 are provided at Table 5

TABLE 5
Antibody 13A6 Sequences

| SEQ ID NO: | | Sequence |
|---|---|---|
| SEQ ID NO: 36 | Antibody VH | ATGAAATGCAGCTGGGTCATCTTCTTCCTG |
| ATGGCAGTGGTTATAGGGGTCAATTCAGA | | |
| GGTTCAGCTGCAGCAGTCTGGGGCTGAGC | | |
| TTGTGAGGCCGGGGGCCTCAGTCAAGTTG | | |
| CCCTGCACAGCTTCTGGCTTTAACATTAAA | | |
| GACGACTATATGAACTGGGTGATGCAGAG | | |
| GCCTGAACGGGGCCTGGAGTGGATTGGAT | | |
| GGATTGATCCTGAGAATGGTGATACTGCA | | |
| TATGCCTCGAAGTTCCAGGGAAAGGCCAC | | |
| TATGACTGCAGACACATCCTCCAACACAGC | | |
| CTACCTGCAGCTCAGCAGCCTGACATCTGA | | |
| GGACACTGCCGTCTATTACTGTACTTCAGG | | |
| TGGTCGTTTCGACTACTGGGGCCAAGGCA | | |
| CCTCTCTCACAGTCTCCTCA | | |
| SEQ ID NO: 37 | Antibody VH | MKCSWVIFFLMAVVIGVNSEVQLQQSGAEL |
| VRPGASVKLPCTASGFNIKDDYMNWVMQR | | |
| PERGLEWIGWIDPENGDTAYASKFQGKAT | | |
| MTADTSSNTAYLQLSSLTSEDTAVYYCTSGG | | |
| RFDYWGQGTSLTVSS | | |
| SEQ ID NO: 38 | Antibody VL | ATGAAGTTGCCTGTTAGGCTGTTGGTGCTG |
| ATGTTCTGGATTCCTGCTTCCAGCAGTGAT | | |
| GTTTTGATGACCCAAACTCCACTCTCCCTG | | |
| CCTGTCAGTCTTGGAGATCAAGCCTCCATC | | |
| TCTTGCAGATCTAGTCAGAGCATTGTACAT | | |
| AGTAATGGAAGCACCTATTTAGAATGGTA | | |
| CCTGCAGAAACCAGGCCAGTCTCCAAAGC | | |
| TCCTGATCTACAAAGTTTCCAACCGCTTITC | | |
| TGGGGTCCCAGACAGGTTCAGTGGCAGTG | | |
| GATCAGGGACAGATTTCACACTCAAGATC | | |
| AACAGAGTGGAGGCTGAGGATCTGGGAG | | |
| TTTATTACTGCTTTCAAGGTTCACATGTTCC | | |
| GTGGACGTTCGGTGGAGGCACCAAGCTGG | | |
| AAATCAAA | | |
| SEQ ID NO: 39 | Antibody VL | MKLPVRLLVLMFWIPASSSDVLMTQTPLSLP |
| VSLGDQASISCRSSQSIVHSNGSTYLEWYLQ | | |
| KPGQSPKLLIYKVSNRFSGVPDRFSGSGSGT | | |
| DFTLKINRVEAEDLGVYYCFQGSHVPWTFG | | |
| GGTKLEIK | | |
| SEQ ID NO: 40 | VH CDR1 | GFNIKDDYMN |
| SEQ ID NO: 41 | VH CDR2 | WIDPENGDTAYASKFQG |
| SEQ ID NO: 42 | VH CDR3 | GGRFDY |
| SEQ ID NO: 43 | VL CDR1 | RSSQSIVHSNGSTYLE |
| SEQ ID NO: 44 | VL CDR2 | KVSNRFS |
| SEQ ID NO: 45 | VL CDR3 | FQGSHVPWT |

Epitope mapping for 13A6 was performed by covalently conjugating synthetic Tau peptides to a Luminex bead set. Purified mAb was incubated with the mixture of peptides on beads, washed, and subsequently incubated with PE-labeled anti-mouse IgG reporter antibodies. The beads were then analyzed on a Luminex FLEXMAP 3D® instrument to detect binding.

Epitope mapping performed on the 13A6 mAb clone revealed binding of a peptide with sequence TREPK (Tau amino acids 220-224) (SEQ ID NO:5). As shown in FIG. 2, the amino acids “PTR” of this epitope are also contained within the peptide selected for MRM analysis: TPSLPTPPTR (SEQ ID NO:1).

To ensure that clone 13A6 equally enriches all Tau isoforms without bias, recoveries of each recombinant isoform of Tau were tested and compared. Recoveries for all six isoforms ranged from 60% to 75% and did not reveal any affinity preference between isoforms.

### Example 3

### Optimization of Immunoaffinity Enrichment Protocol

Following antibody selection, multiple parameters of the Tau immunoaffinity binding, wash, elution, and digestion steps were evaluated in order to define immunoaffinity enrichment conditions that provided maximum and reproducible signal intensity.

The quantity of beads required for maximal Tau recovery from 1 ml CSF was tested by titration from 0.2 to 0.0125 mg beads, with maximal recovery observed in all samples containing at least 0.05 mg beads. All sample preparation procedures were developed in 1.8 mL 96-well plates to maximize throughput allow CSF sample volumes as high as 1 ml. For liquid additions and transfers, the use of 96-well plates compatible with 96 head liquid handling robotics rather than individual tubes was critical to obtaining the reproducibility and throughput to support clinical studies

Digestion efficiency was evaluated by measuring the signal of the TPSLPTPPTR (SEQ ID NO: 1) peptide over the course of a 24 hour digestion of CSF using 0.5 μg trypsin per sample. Product formation was essentially complete at 5 hours, with no reduction in signal intensity observed up to 24 hours, indicating no over-digestion.

Although digestion of Tau was found to be efficient when performed directly on the magnetic beads without a protein elution step, the use of an acidic elution step followed by SpeedVac solvent evaporation was found to be preferable as it significantly reduced background noise approximately three fold, and therefore LOQ.

Another feature of this assay crucial for the analysis of clinical samples is the use of an isotope labeled internal standard (IS), which is used to correct for differences in recovery during sample extraction/processing. Heavy-isotope labeled (¹³C-Lys and ¹³C-Arg) recombinant Tau was spiked into CSF samples prior to immunoaffinity purification and subsequent digestion and analysis (FIG. 1). The value of a protein IS over a peptide IS is that it can be added at the beginning of the sample preparation procedure and therefore accounts for variations of both immunoaffinity enrichment and digestion. The suitability of using this single isoform recombinant protein as an internal standard for all endogenous isoforms was tested to ensure there was no bias in immunoaffinity enrichment. A mixture of IS Tau and endogenous Tau (using postmortem CSF) was prepared and the ratio of light to heavy compared with or without immunoaffinity enrichment (n=4). A t-test could not detect a change in light/heavy Tau ratio, indicating that the recombinant Tau IS is not distinguished from endogenous Tau using our IA method.

The use of micro flow chromatography combined with a microfluidic device provides enhanced sensitivity with the robustness required for clinical assays. Zhou et al. (2013) Rapid Commun. Mass Spectrom. 27:1294-302. The sensitivity gained from relatively high starting volumes of CSF and ultrasensitive detection allows the quantitation of low abundant sequences including low abundant PTMs. The optimized sample preparation procedure was designed for throughput with the majority of steps performed in 96 well plates and with automation. The quantity of beads required for optimal Tau recovery was determined to be at least 0.05 mg beads; the trypsin digestion was found to be optimal using 0.5 μg trypsin per sample overnight. The LC/MS cycle time is 13 min and is therefore capable of analyzing 96 samples in less than 24 hours.

### Example 4

### LC-MS/MS Profiling of Total-Tau in CSF from Human Postmortem CSF

Immunoaffinity purified and digested Tau from human postmortem CSF was analyzed by full scan MS with data dependent MS/MS for identification of peptides. Up to thirty four (34) distinct Tau peptides were observed in the postmortem samples. Table 6 provides a summary of the Tau peptides detected in the postmortem samples. Both unmodified and modified peptides were observed, with peptides of interest indicated in bold italics. Table 7 assigns SEQ ID NOS: to each of the Tau peptides.

Because Tau present in CSF is known to have heterogeneous mass, due to multiple isoforms and post-translational modifications, molar units more accurately describe Tau levels in CSF. Therefore, concentration units for our mass spectrometry based assay were expressed in picomolar (pM) quantities rather than the standard pg/mL units typically used by immunoassays.

TABLE 6
Tau Peptides Identified in Immunopreciptates of post-Mortem
Cerebral Spinal Fluid

| | | Mass | |
|---|---|---|---|
| | Mascot | Error | Peak |
| Peptide Sequence | Score | (ppm) | Intensity |
| YVSSVTPR | 33.88 | −1.66 | 4.1E+04 |
| VQIVYKPVDLSK | 39 | −1.49 | 6.3E+04 |
| VAVVRT(Phos)PPK | 23.6 | −1.44 | 8.5E+04 |
| [symbol] | 44.55 | −2.85 | 8.7E+06 |
| [symbol] [symbol] | 20.83 | −1.28 | 1.5E+05 |
| TPPSSGEPPK | 36.39 | −6.98 | 2.0E+04 |
| TPPAPKT(Phos)*PPSSGEPPK | 38.68 | −0.07 | 1.7E+04 |
| TDHGAEIVYK | 29.48 | −0.8 | 8.6E+04 |
| [symbol] [symbol] | 71.57 | −0.97 | 2.3E+04 |
| [symbol] [symbol] [symbol] | 50.61 | −0.45 | 2.6E+04 |
| STPTAEAEEAGIGDTPSLEDEAAGHVTQAR | 71.31 | −0.59 | 2.1E+05 |
| STPT(Phos)*AEAEEAGIGDTPSLEDEAAGHVTQAR | 69.63 | −1.23 | 6.0E+04 |
| SPVVSGDTSPR | 32.35 | −1.45 | 7.5E+04 |
| SPVVSGDTS(Phos)PR | 60.35 | −0.61 | 4.7E+04 |
| SGYSSPGSPGTPGSR | 53.49 | −0.9 | 4.7E+06 |
| SGYSSPGS(Phos)PGTPGSR | 59.72 | 0.15 | 1.6E+05 |
| QEFEVMEDHAGTYGLGDR | 57.91 | −2.77 | 6.5E+05 |
| QAAAQPHTEIPEGTTAEEAGIGDTPSLEDEAAGHVTQAR | 54.19 | −0.97 | 8.4E+04 |
| [symbol] [symbol] | 64.24 | −1.78 | 4.1E+06 |
| [symbol] [symbol] | 46.58 | −0.41 | 1.4E+06 |
| LDLSNVQSK | 47.48 | −1.63 | 3.2E+05 |
| KLDLSNVQSK | 61.89 | −1.14 | 1.7E+05 |
| KDQGGYTMHQDQEGDTDAGLK | 68.5 | −0.43 | 6.1E+04 |
| KDQGGYTM(Ox)HQDQEGDTDAGLK | 52.64 | −0.31 | 3.9E+04 |
| IGSTENLK | 32.38 | −0.47 | 5.0E+04 |
| [symbol] [symbol] | 66.24 | −0.29 | 9.1E+04 |
| ESPLQTPTEDGSEEPGSETSDAK | 61.28 | −1.32 | 6.4E+05 |
| ESPLQTPTEDGSEEPGS(Phos)*ETSDAK | 51.35 | −0.77 | 2.9E+04 |
| DQGGYTMHQDQEGDTDAGLKES(Phos)PLQTPTEDGSEEPGSETSDAK | 42.35 | −0.28 | 3.5E+04 |
| DQGGYTMHQDQEGDTDAGLK | 70.13 | −1.68 | 2.7E+04 |
| DQGGYTMHQDQEGDTDAGLK | 53.67 | −0.94 | 1.6E+05 |
| DQGGYTM(Ox)HQDQEGDTDAGLK | 73.72 | −0.8 | 6.4E+04 |
| AEEAGIGDTPSLEDEAAGHVTQAR | 46.09 | −0.05 | 1.8E+05 |
| AAFPGAPGEGPEAR | 22.15 | −1.87 | 7.8E+04 |

(Ox) = Oxidation, (Phos) = Phosphorylation, * ambiguous localization of modification

TABLE 7
Summary of Tau Peptide SEQ ID NOS:

| SEQ | |
|---|---|
| ID | |
| NO: | Peptide Sequence |
| 1 | [symbol] |
| 2 | [symbol] [symbol] |
| 3 | [symbol] [symbol] |
| 4 | [symbol] [symbol] |
| 6 | YVSSVTPR |
| 7 | VQIVYKPVDLSK |
| 8 | VAVVRT(Phos)PPK |
| 9 | [symbol] [symbol] |
| 10 | TPPSSGEPPK |
| 11 | TPPAPKT(Phos)*PPSSGEPPK |
| 12 | TDHGAEIVYK |
| 13 | [symbol] [symbol] |
| 14 | [symbol] [symbol] [symbol] |
| 15 | STPTAEAEEAGIGDTPSLEDEAAGHVTQAR |
| 16 | STPT(Phos)*AEAEEAGIGDTPSLEDEAAGHVTQAR |
| 17 | SPVVSGDTSPR |
| 18 | SPVVSGDTS(Phos)PR |
| 19 | SGYSSPGSPGTPGSR |
| 20 | SGYSSPGS(Phos)PGTPGSR |
| 21 | QEFEVMEDHAGTYGLGDR |
| 22 | QAAAQPHTEIPEGTTAEEAGIGDTPSLEDEAAGHVTQAR |
| 23 | [symbol] [symbol] |
| 24 | LDLSNVQSK |
| 25 | KLDLSNVQSK |
| 26 | KDQGGYTMHQDQEGDTDAGLK |
| 27 | KDQGGYTM(Ox)HQDQEGDTDAGLK |
| 28 | IGSTENLK |
| 29 | ESPLQTPTEDGSEEPGSETSDAK |
| 30 | ESPLQTPTEDGSEEPGS(Phos)*ETSDAK |
| 31 | DQGGYTMHQDQEGDTDAGLKES(Phos) |
| | PLQTPTEDGSEEPGSETSDAK |
| 32 | DQGGYTMHQDQEGDTDAGLK |
| 33 | DQGGYTM(Ox)HQDQEGDTDAGLK |
| 34 | AEEAGIGDTPSLEDEAAGHVTQAR |
| 35 | AAFPGAPGEGPEAR |

All three peptides identified using the in silico technique described in Example 1 as potential total-Tau surrogate peptides were detected in the postmortem samples. The Tau peptide comprising the amino acid sequence set forth in SEQ ID NO: 1 (TPSLPTPPTR) was detected in the human post mortem CSF samples and found to be only minimally phosphorylated.

The Tau peptide comprising the amino acid IGSLDNITHVPGGGNK (SEQ ID NO:3), was observed in some samples with a missed cleavage IGSLDNITHVPGGGNKK (data not shown) (SEQ ID NO:4), suggesting that trypsin digestion may be problematic for this peptide. The other candidate surrogate Tau peptide, LQTAPVPMPDLK (SEQ ID NO:2), was also detected, but it was observed to be oxidized with a peak intensity roughly ⅓ of the unoxidized peptide. Both of these observations suggest that surrogate peptides comprising the amino acid sequences set forth in SEQ ID NOs: 2 and 3 are less suitable for use as a surrogate peptide than the surrogate peptide having an amino acid sequence set forth in SEQ ID NO: 1. In comparison, several of the other identified Tau peptides were observed to be phosphorylated to a much greater extent, including SPVVSGDTSPR (SEQ ID NOs:17 and 18) and the isoform specific peptide STPTAEDVTAPLVDEGAPGK (SEQ ID NOs: 13 and 14).

The tryptic Tau peptide comprising the amino acid sequence set forth in SEQ ID NO:1 was identified as an optimal surrogate peptide for the development of a total-Tau IA-MS assay.

### Example 5

### Fit-for-Purpose Analytical Validation

Limit of quantitation for this assay was established by using recombinant standard spiked and diluted into purchased CSF tested to have no detectable Tau. The lower limit of quantitation (LLOQ) of the assay was calculated using 1 mL of CSF to be 0.25 pM, where both error and CV fall outside of our 20% criteria.

Intra-assay reproducibility was determined by measuring 6 single-use aliquots for each QC level in a single run and inter-assay reproducibility was determined by measuring a single-use aliquot for each QC level on 6 separate days over the course of 2 weeks. Values for mean concentration, standard deviation, and % coefficient of variation for each QC level are displayed in Table 8.

TABLE 8
Inter- and Intra- day reproducibility values
(pM) for CSF Tau quality control samples.

| | Interday Precision | | | Intraday Precision | | |
|---|---|---|---|---|---|---|
| | Mean | SD | % CV | Mean | SD | % CV |
| Low | 2.0 | 0.4 | 18.9 | 2.0 | 0.2 | 8.1 |
| Medium | 17.1 | 1.5 | 9.0 | 15.3 | 0.5 | 3.2 |
| High | 40.7 | 3.2 | 7.8 | 38.9 | 1.5 | 3.8 |

The reproducibility values summarized in Table 8 are considered acceptable for all protein QC levels. Dilution linearity experiments revealed that dilutions of CSF 1:16 (62.5 μl CSF diluted to 1 mL with PBS) were found to be acceptable (<20% change from undiluted). Spike recovery at three concentration levels found less than 10% difference of the observed from the expected value at all levels.

The assay development was subjected to a fit-for-purpose validation which indicated that it had excellent precision, spike recovery and the sufficient sensitivity to measure Tau levels in all human patients studied. There was an excellent correlation (r²=0.950) between Tau concentrations determined by the IA-MS method.

### Example 6

### Comparison of IA-MS Assay and Immunoassay Using Patient CSF Samples

CSF samples from age matched normal and AD subjects were analyzed for Tau levels using this IA-MS assay as well as a commercially available immunoassay kit, the Human Total Tau kit (K151LAE) from Meso Scale Discovery (MSD) (Rockville, Md., USA).

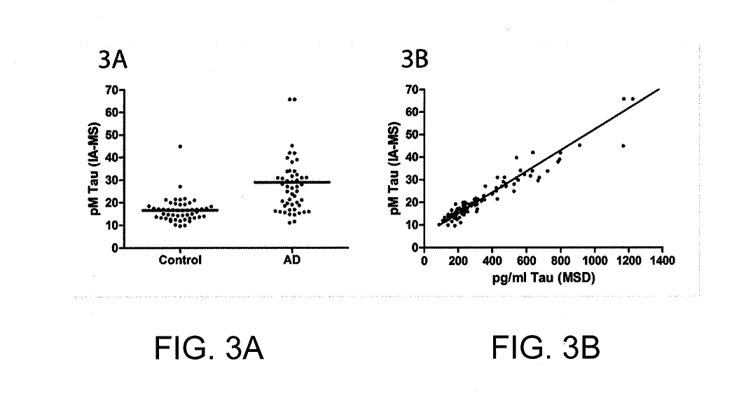

To conserve CSF volume, 150 μl of CSF was analyzed by IA-MS. Tau levels were measurable in all samples even using a 6.7 dilution factor. As shown in FIG. 3A, IA-MS identified a significant difference (P<0.0001) in Tau levels between normal and AD groups. The total-Tau CSF values for the two assays are highly correlated (r²=0.950) as demonstrated in FIG. 3B. Conversion of the IA-MS data from pM to pg/ml using an estimated average protein MW of 40 kDa yields a correlation slope of 1.8, which may reasonably be accounted for by differences in the calibration standards used in this assay and those included in the MSD kit.

CSF samples were incubated with anti-Tau monoclonal antibodies covalently conjugated to magnetic beads. Bound Tau was eluted off the beads, dried and digested into peptides with trypsin. Tau proteotrypic peptides were quantified using a Waters Xevo TQ-S with a Trizaic nanoflow UPLC microfluidic device. Further characterization of Tau peptides was done by full scan MS using a Thermo Orbitrap with nanoAcquity UPLC system. In addition, CSF samples from a cohort of age matched controls and subjects with AD were analyzed by the IA-MS method and by a commercially available immunoassay.

Tau values were found to correlate between the two assays but some scatter was seen around the correlation line. This may indicate that, while both assays seem to recognize the same analyte, there may be some subtle differences in specificity, which is also reflected by the slight change in fold-change between AD and controls measured by both assays. Therefore, we have provided a very useful tool that can be used in the better understanding and qualification of immunoassays. This is of particular value for current collaborative efforts, such as with the Global Consortium for the Standardization of CSF Biomarkers (GCSB) (Dufield & Radabaugh (2012) Methods 56:236-45), which is working to establish standardized reference methods for the measurement of clinical AD biomarkers.

As shown herein, combining immunoaffinity extraction with microflow LC-MS/MS analysis is an effective approach for the development of a highly selective assay to measure total-Tau and potentially other post-translational modifications of Tau. As expected, Tau was found to be elevated in AD patients by approximately 2-fold. The IA-MS assay for CSF Tau presented here provides for the first time a mass spectrometric-based method for the measurement of total Tau in human CSF.

Patents, patent applications, publications, product descriptions, and protocols are cited throughout this application, the disclosures of which are incorporated herein by reference in their entireties for all purposes. All references cited herein are incorporated by reference to the same extent as if each individual publication, database entry (e.g. GenBank sequences or GeneID entries), patent application, or patent, was specifically and individually indicated to be incorporated by reference. Citation of the references herein is not intended as an admission that the reference is pertinent prior art, nor does it constitute any admission as to the contents or date of these publications or documents.

Sequences disclosed herein are provided at Table 9.

TABLE 9
Sequences

| SEQ | | |
|---|---|---|
| ID: | Description | Sequence |
| 1 | Tau peptide | TPSLPTPPTR |
| 2 | Tau peptide | LQTAPVPMPDLK |
| 3 | Tau peptide | IGSLDNITHVPGGGNK |
| 4 | Tau peptide | IGSLDNITHVPGGGNKK |
| 5 | 13A6 epitope | TREPK |
| 6 | Tau peptide | YVSSVTPR |
| 7 | Tau peptide | VQIVYKPVDLSK |
| 8 | Tau peptide | VAVVRT(Phos)PPK |
| 9 | Tau peptide | TPS(Phos)LPTPPTR |
| 10 | Tau peptide | TPPSSGEPPK |
| 11 | Tau peptide | TPPAPKT(Phos)*PPSSGEPPK |
| 12 | Tau peptide | TDHGAEIVYK |
| 13 | Tau peptide | STPTAEDVTAPLVDEGAPGK |
| 14 | Tau peptide | ST(Phos)*PTAEDVTAPLVDEGAPGK |
| 15 | Tau peptide | STPTAEAEEAGIGDTPSLEDEAAGHVTQAR |
| 16 | Tau peptide | STPT(Phos)*AEAEEAGIGDTPSLEDEAAGHVTQAR |
| 17 | Tau peptide | SPVVSGDTSPR |
| 18 | Tau peptide | SPVVSGDTS(Phos)PR |
| 19 | Tau peptide | SGYSSPGSPGTPGSR |
| 20 | Tau peptide | SGYSSPGS(Phos)PGTPGSR |
| 21 | Tau peptide | QEFEVMEDHAGTYGLGDR |
| 22 | Tau peptide | QAAAQPHTEIPEGTTAEEAGIGDTPSLEDEAAGHVTQAR |
| 23 | Tau peptide | LQTAPVPM(Ox)PDLK |
| 24 | Tau peptide | LDLSNVQSK |
| 25 | Tau peptide | KLDLSNVQSK |
| 26 | Tau peptide | KDQGGYTMHQDQEGDTDAGLK |
| 27 | Tau peptide | KDQGGYTM(Ox)HQDQEGDTDAGLK |
| 28 | Tau peptide | IGSTENLK |
| 29 | Tau peptide | ESPLQTPTEDGSEEPGSETSDAK |
| 30 | Tau peptide | ESPLQTPTEDGSEEPGS(Phos)*ETSDAK |
| 31 | Tau peptide | DQGGYTMHQDQEGDTDAGLKES(Phos)PLQTPTEDGSEEPGSE |
| | | TSDAK |
| 32 | Tau peptide | DQGGYTMHQDQEGDTDAGLK |
| 33 | Tau peptide | DQGGYTM(Ox)HQDQEGDTDAGLK |
| 34 | Tau peptide | AEEAGIGDTPSLEDEAAGHVTQAR |
| 35 | Tau peptide | AAFPGAPGEGPEAR |
| 36 | VH DNA | ATGAAATGCAGCTGGGTCATCTTCTTCCTGATGGCAGTGGT |
| | | TATAGGGGTCAATTCAGAGGTTCAGCTGCAGCAGTCTGGG |
| | | GCTGAGCTTGTGAGGCCGGGGGCCTCAGTCAAGTTGCCCTG |
| | | CACAGCTTCTGGCTTTAACATTAAAGACGACTATATGAACT |
| | | GGGTGATGCAGAGGCCTGAACGGGGCCTGGAGTGGATTGG |
| | | ATGGATTGATCCTGAGAATGGTGATACTGCATATGCCTCGA |
| | | AGTTCCAGGGAAAGGCCACTATGACTGCAGACACATCCTC |
| | | CAACACAGCCTACCTGCAGCTCAGCAGCCTGACATCTGAG |
| | | GACACTGCCGTCTATTACTGTACTTCAGGTGGTCGTTTCGA |
| | | CTACTGGGGCCAAGGCACCTCTCTCACAGTCTCCTCA |
| 37 | VH protein | MKCSWVIFFLMAVVIGVNSEVQLQQSGAELVRPGASVKLPCT |
| | | ASGFNIKDDYMNWVMQRPERGLEWIGWIDPENGDTAYASKF |
| | | QGKATMTADTSSNTAYLQLSSLTSEDTAVYYCTSGGRFDYW |
| | | GQGTSLTVSS |
| 38 | VL DNA | ATGAAGTTGCCTGTTAGGCTGTTGGTGCTGATGTTCTGGAT |
| | | TCCTGCTTCCAGCAGTGATGTTTTGATGACCCAAACTCCAC |
| | | TCTCCCTGCCTGTCAGTCTTGGAGATCAAGCCTCCATCTCTT |
| | | GCAGATCTAGTCAGAGCATTGTACATAGTAATGGAAGCAC |
| | | CTATTTAGAATGGTACCTGCAGAAACCAGGCCAGTCTCCAA |
| | | AGCTCCTGATCTACAAAGTTTCCAACCGCTTTTCTGGGGTC |
| | | CCAGACAGGTTCAGTGGCAGTGGATCAGGGACAGATTTCA |
| | | CACTCAAGATCAACAGAGTGGAGGCTGAGGATCTGGGAGT |
| | | TTATTACTGCTTTCAAGGTTCACATGTTCCGTGGACGTTCG |
| | | GTGGAGGCACCAAGCTGGAAATCAAA |
| 39 | VL protein | MKLPVRLLVLMFWIPASSSDVLMTQTPLSLPVSLGDQASISCR |
| | | SSQSIVHSNGSTYLEWYLQKPGQSPKLLIYKVSNRFSGVPDRF |
| | | SGSGSGTDFTLKINRVEAEDLGVYYCFQGSHVPWTFGGGTKL |
| | | EIK |
| 40 | VH CDR1 | GFNIKDDYMN |
| 41 | VH CDR2 | WIDPENGDTAYASKFQG |
| 42 | VH CDR3 | GGRFDY |
| 43 | VL CDR1 | RSSQSIVHSNGSTYLE |
| 44 | VL CDR2 | KVSNRFS |
| 45 | VL CDR3 | FQGSHVPWT |
| 46 | AT120 | PPTREPK |
| | epitope | |

What is claimed is:

1. A method for measuring Tau protein in a biological sample comprising the steps of:
a. providing a biological sample suspected of comprising Tau protein;
b. adding an isotope labeled internal standard Tau protein to the sample to produce a spiked sample;
c. contacting the spiked sample with an anti-Tau antibody that is covalently conjugated to solid phase particles;
d. maintaining the sample produced in step c) under conditions suitable to allow the anti-Tau antibody to bind to Tau protein present in the sample;
e. washing the particles;
f. eluting the Tau protein from the particles;
g. contacting the protein recovered in step e) with trypsin under conditions suitable to digest the Tau protein into an analyte sample comprising a composition of peptides, and;
h. performing mass spectroscopic analysis to detect and measure the concentration of a surrogate Tau peptide present in the analyte sample.

2. The method of claim 1, wherein the biological sample is cerebral spinal fluid (CSF), the isotope labeled internal standard is a heavy-isotope labeled recombinant human Tau protein, and the mass spectroscopic analysis is performed using a Trizaic nanoTile UPLC microfluidic device.

3. The method of claim 1, wherein the anti-Tau antibody binds to an epitope that overlaps with the surrogate Tau peptide.

4. The method of claim 3, wherein the anti-Tau antibody binds to an epitope comprising the amino acid sequence set forth in SEQ ID NO: 5.

5. The method of claim 4, wherein the anti-Tau antibody comprises a variable heavy chain comprising the amino acid sequence set forth in SEQ ID NO: 37 and a variable light chain comprising the amino acid sequence set forth in SEQ ID NO: 39.

6. The method of claim 4, wherein the anti-Tau antibody comprises:
a) a variable heavy chain comprising a CDR1 as set forth in SEQ ID NO: 40, a CDR2 as set forth in SEQ ID NO: 41 and a CDR3 asset forth in SEQ ID NO: 42; and
b) a variable light chain comprising a CDR1 as set forth in SEQ ID NO: 43, a CDR2 as set forth in SEQ ID NO:44 and a CDR3 as set forth in SEQ ID NO:45.

7. The method of claim 1, wherein the solid phase particles are magnetic beads.

8. The method of claim 1 wherein the elution (e) is performed under acidic conditions, and further wherein the method optionally comprises a solvent evaporation or neutralization step.

9. The method of claim 1, wherein the trypsin digestion (0 is performed overnight at 37° C. at pH7.5-8.5.

10. The method of claim 1, wherein the surrogate Tau peptide comprises the amino acid sequence set forth in SEQ ID NO:1.

11. A surrogate Tau peptide comprising the amino acid sequence set forth in SEQ ID NO:1.
